(19) United States
(12) Patent Application Publication
(10) Pub. No.: US 2017/0048628 A1
(43) Pub. Date: Feb. 16, 2017

(54) Laser-activatable variable-length ossicular prosthesis

(71) Applicant: Heinz Kurz GmbH Medizintechnik, Dusslingen (DE)

(72) Inventors: John T. McElveen, Raleigh, NC (US); Uwe Steinhardt, Hirrlingen (DE)

(21) Appl. No.: 15/232,097

(22) Filed: Aug. 9, 2016

(30) Foreign Application Priority Data
Aug. 10, 2015 (DE) 102015113138.2

(51) Int. Cl. H04R 25/00; A61F 2/18

(52) U.S. Cl. CPC H04R 25/606; A61F 2/18; A61F 2002/183

(57) ABSTRACT

An ossicular prosthesis includes a first fastening element formed as a perforated tympanic membrane top plate and a second fastening element for coupling to a stapes of an inner ear of a patient under treatment. The ossicular prosthesis includes a connecting element having an adjusting device for adjusting the axial length. The connecting element includes two partial branches that extend symmetrically with respect to the longitudinal axis, are extendable, compressible or both, can be permanently plastically deformed, and are folded in multiple loops. The loops are made from a material having a memory effect. Each of the loops of one of the partial branches has an activation tab that is thermally conductively attached to the most radially remote outer area, extends radially away from the loop, and has an activation surface perpendicular to the longitudinal axis. The activation surfaces have a successively increasing radial distance in the axial direction.

### CROSS-REFERENCE TO A RELATED APPLICATION

The invention described and claimed hereinbelow is also described in German Priority Document DE 10 2015 113 138.2, filed on Aug. 10, 2015. The German Priority Document, the subject matter of which is incorporated herein by reference, provides the basis for a claim of priority of invention under 35 U.S.C. 119(a)-(d).

### BACKGROUND OF THE INVENTION

The invention relates to a an ossicular prosthesis that replaces or bridges at least one component of the human ossicular chain. The ossicular prosthesis comprises, at one end, a first fastening element designed as a perforated top plate for placement against the tympanic membrane and, at the other end, a second fastening element for mechanically connecting the prosthesis to the head of stapes or the base of stapes. An elongate connecting element interconnects the two fastening elements in a sound-conducting manner. The connecting element includes an adjusting device for adjusting the axial length of the ossicular prosthesis in the axial direction of the elongate connecting element. The first fastening element is mechanically rigidly connected to one end of the connecting element, and the second fastening element is mechanically rigidly connected to the other, axially opposite, end of the connecting element. The adjusting device includes at least two partial branches which extend symmetrically with respect to the longitudinal axis of the connecting element, are extendable and/or compressible in the axial direction, are permanently plastically deformable, and are folded into multiple loops transverse to the longitudinal axis, in a serpentine, meandering, or accordion-like manner, before being deformed, and wherein parts of the ossicular prosthesis are made from a material having a memory effect.

Ossicular prostheses of this type are known, see, for example, from DE 10 2009 016 468 B3.

Ossicular prostheses are used to conduct sound or the sound signal from the tympanic membrane to the inner ear in cases in which the ossicles of the human middle ear are missing or damaged, in entirety or in part. The ossicular prosthesis has two ends. Depending on the specific circumstances, one end of the ossicular prosthesis is fastened to the tympanic membrane, e.g. using a top plate, and the other end of the ossicular prosthesis is fastened, e.g. to the stapes of the human ossicular chain, or it is inserted directly into the inner ear. In the case of the known ossicular prostheses (e.g., DE 10 2009 016 468 B3), sound conduction or signal transmission between the tympanic membrane and the inner ear is limited in many cases, because these known ossicular prostheses cannot not fully replace the natural anatomical formations of the ossicular chain.

Three types of ossicular prostheses which are used particularly frequently are stapes prostheses, partial prostheses, and total prostheses. Stapes prostheses are fixed to the incus and extend via a piston into the inner ear. Partial prostheses typically bear via a top plate against the tympanic membrane and establish a connection to the head of the stapes. Total prostheses connect the tympanic membrane to the base of stapes.

One major problem which arises in every case of reconstructing the human ossicular chain involves selecting the correct length of prosthesis. The lengths that are required vary within a range of several millimeters, due to differences in anatomy. When an ossicular prosthesis is surgically implanted, it is therefore necessary to have on hand a sufficiently large selection of prostheses having different axial lengths, or it must be possible to reduce the maximum starting length of the ossicular prostheses to the final axial length that is required for the particular patient's requirement.

In terms of a postsurgical position adjustment, ossicular prostheses are advantageous which are composed, in entirety or in part, in particular in the area of one of the fastening elements, of a material having a memory effect or superelastic properties, preferably being composed of Nitinol, as is known, for example, from WO 02/069850 A1 or U.S. Pat. No. 6,554,861 B2. This effect also is utilized, for example, in the case of an ossicular prosthesis of the type described in DE 10 2007 008 851 B3. In this case, an initially half-open bight which, as a fastening element, is pulled over the incus or the manubrium of malleus, during implantation is closed quasi contactlessly about the corresponding ossicle and so the prosthesis is fixed thereon. In a similar context, U.S. Pat. No. 6,197,060 B1, for instance, describes an article for self-securing by means of thermal activation of a prosthetic section using a laser.

A device, the length of which is adjustable with the aid of a clamping effect, for mechanically coupling the driver of an active hearing aid to a coupling point of the ossicular chain, is described in DE 199 48 375 A1.

WO 92/18066 A1 describes a self-adjusting, passive ossicular prosthesis which includes a spring mechanism, but which is complicated and very costly to manufacture, in the connection between the first and second fastening elements. The spring mechanism makes it possible to continuously change the axial length of the prosthesis depending on the relative position of the fastening points in the middle ear. It is not possible, therefore, to attain a fixed, reproducibly exact length of the prosthesis even though the length is retained after the prosthesis has been surgically implanted in the middle ear. In addition, due to its very special mechanical and geometric design, the known prosthesis requires a great deal of space in the middle ear, thereby rendering it entirely unusable in many cases due to the unique features of a particular patient. In addition, due to the design, a considerable amount of permanent pressure builds up between the two fastening points in the middle ear after implantation, which does not exactly promote healing after surgery and often eventually results in postsurgical complications.

A passive ossicular prosthesis having an axial length which can be varied within certain limits during surgery is described in DE 39 01 796 A1. In that case, the length is changed by bending the connecting element, which is designed as a thin gold wire. Consequently, handling is complicated and relatively inaccurate, thereby rendering it challenging if not impossible to attain the desired exact axial length of the ossicular prosthesis. In addition, the result that is attained using this technique is not always reproducible, and, once the connecting element has been bent, it also is possible for the adjusted axial length of the ossicular prosthesis to change because the connecting element springs back.

EP 0 998 884 A2 describes a passive ossicular prosthesis, in which the first connecting element, which is designed as an elongate shank, is inserted through a through-bore of the first fastening element, which is designed as a top plate, until a desired shank length between the first and second fastening elements is attained. The shank is then fixed in this position by constricting the through-bore in the top plate, and the section of the shank that extends past the top plate is trimmed off. One therefore easily obtains a prosthesis which has the particular length that is desired or required, and which remains exactly the same, after surgery in particular.

DE 10 2005 010 705 B3 makes known an ossicular prosthesis in which an intraoperative variability of the prosthesis length is attained by virtue of the fact that the elongate connecting element is designed in the form of a ball chain. During surgery, the ball chain is inserted through a receiving opening in the first fastening element via a certain number of balls. The ball chain is then fixed in the receiving opening of the fastening element using resilient segment elements which clamp onto either side of the ball chain, and the overhanging part of the ball chain which extends through the receiving opening is cut off, and so the prosthesis ultimately has exactly the desired axial length. In a similar manner, the length variability also is attained using an ossicular prosthesis as described in DE 20 2005 015 944 U1, in which case a trimmable ball chain is likewise used as the connecting element, but the receptacle in the first fastening element has a different design.

A further passive ossicular prosthesis having an intraoperatively variable axial length is described in U.S. Pat. No. 3,710,399. In that case, a two-piece connecting element is used between the two fastening elements. The two-piece connecting element comprises two parallel, straight wire pieces, one of which extends away from the first fastening element, and the second of which extends away from the second fastening element. The two wire pieces are connected to the particular other wire piece using wire loops at their ends, or they can be inserted into a type of connecting coupling having two parallel longitudinal bores for the two wire pieces. In the first case, it is not possible, however, to exactly adjust the fixing position and, therefore, the relative position of the two wire pieces, thereby making it challenging if not impossible to adjust the length of the prosthesis in an exact and reproducible manner. In the second case, once the wire pieces have been inserted into the connecting coupling, the relative positions of the wire pieces can tilt, flex, or become displaced, thereby likewise making it difficult or impossible to exactly adjust the axial length of the prosthesis.

Another technique for adjusting the length of a passive ossicular prosthesis is made known in DE 10 2005 027 215 A1. The prosthesis described therein is designed exclusively for use in the situation of stapes surgery, and so a plunger-shaped piston is always provided as the second fastening element. A receiving mechanism is located in this piston, into which the shank-shaped connecting element will be inserted in the axial direction. Leaf springs which are spread radially apart by the connecting element have an arresting effect in a desired relative position between the connecting element and the second fastening element. Aside from the fact that an exactly reproducible adjustment of a desired axial length of the prosthesis is therefore not always guaranteed, the scope of application of this ossicular prosthesis is limited to surgery of the stapes, in the case of which a direct connection to the inner ear is attained via the piston. However, if a bell, piston, clip, or flat shoe is used as the second fastening part, for connection to another part of the ossicular chain, then this known prosthesis is not usable. If the intention is to form a related receiving mechanism in the second fastening part, then, due to geometry, it functions only in a piston and never in bell, flat shoe, or even in a clip.

The variable-length ossicular prosthesis described in DE 297 22 084 U1 likewise covers a scope of application that is greatly limited. Instead of a shank-shaped connecting element, this variable-length prosthesis includes three segment elements that can be snapped off in the manner of a stand, one end of which leads into a bell-shaped or piston-shaped body for fastening to the stapes and the other end of which leads into a top plate for placement against the tympanic membrane. This design can be used exclusively in combination with a plate-type fastening element, i.e., only when coupling to the tympanic membrane. Another disadvantage of this prosthesis is the fact that it does not include a defined shank as the connecting element between the two fastening elements and so the prosthesis can shift or flex transverse to the longitudinal axis of the prosthesis if axial force is not introduced absolutely exactly.

The ossicular prosthesis described in U.S. Pat. No. 5,554,188, likewise comprises a connecting element which is designed as a two-piece shank, in which the first, rod-shaped section is inserted into a receiving bore of the second section, which is designed as a receiving part, and is displaceable axially in the bore. To attain a desired axial length of the prosthesis, the rod-shaped first section is trimmed from a maximum starting length to a suitable final length and is inserted into the second section until it stops. By suitably designing the inner diameter of the receiving bore relative to the outer diameter of the first section, a frictional clamping of the first and second sections should bring about a certain fixation of the prosthesis length, the actual fixation being attained by virtue of the fact that the parts of the prosthesis that can move in opposite directions are unable to move very far apart from one another after surgical implantation in the middle ear, due to their being stopped at the two fastening points. It is therefore impossible to ensure that a length of the prosthesis will always remain exactly the same.

In the case of the passive ossicular prosthesis described in US 2003/0097178 A1, the receiving part also includes a cavity which is open in the direction toward the insertion part and extends in the axial direction of the connecting element. The connecting element is designed to have a variable length in the axial direction between the receiving part and the insertion part, and the specific axial lengths of the connecting element of a specific ossicular prosthesis are fixed by clamping the insertion part to the receiving part in a desired relative coaxial insertion position. In principle, it is therefore possible to attain a desired, defined length of the prosthesis even before it is clamped between the two fastening points. This length also is fixedly retained after surgery, e.g., by inserting a second fastening element, which is designed as a piston, through a perforated base of the stapes.

DE 20 2007 012 217 U1 makes known an ossicular prosthesis, in which the clamping force, in the clamped state, between the receiving part and the insertion part is selected to be considerably greater than the maximum external forces which occur naturally in the middle ear in the region of the ossicles. This makes it possible to vary the length of the passive ossicular prosthesis “in situ” or intraoperatively, and relatively large selections of prostheses having different lengths do not need to be kept on hand during every surgical procedure. In addition, it is simple to adjust the particular length of the prosthesis that is desired, and, therefore, the handling is likewise simple. Due to the selection of the clamping force described above, subsequent, postsurgical, undesirable changes in length and/or position of the prosthesis are reliably prevented. In addition, this known ossicular prosthesis can be used universally in all conceivable types of couplings in the middle ear space, and it is not limited to a certain class of surgeries, whereas, e.g., the prosthesis described according to above-cited DE 10 2005 027 215 A1 can only be used exclusively in the situation of stapes surgery. However, these advantages are attained by means of a relatively complicated, mechanical design of the adjusting device in the connecting element of the prosthesis, a level of production outlay which is considerable by nature, and resultant relatively high manufacturing costs. Finally, DE 10 2009 016 468 B3 cited at the outset discloses an ossicular prosthesis in which the number of different prostheses which must be kept on hand intraoperatively can be greatly reduced, while ensuring that the prosthesis can be optimally adapted for a specific case and replacing the complex design of the adjusting device of the ossicular prosthesis made known in DE 10 2005 027 215 A1 by a substantially simpler mechanical design. This is achieved by way of the adjusting device including at least two partial branches which extend symmetrically with respect to the longitudinal axis of the connecting element, are extendable and/or compressible in the axial direction, are permanently plastically deformable and are folded into multiple loops transverse to the longitudinal axis before being deformed. This known ossicular prosthesis is intraoperatively set to a certain desired length by mechanically compressing or pulling apart one or multiple loops in the adjusting device.

### SUMMARY OF THE INVENTION

The present invention overcomes the shortcomings of known arts, such as those mentioned above.

To that end, the present invention improves and modifies a generic, variable-length, middle-ear prosthesis of the type mentioned at the outset using simple technical means, in a simple and cost-effective manner such that the length of the ossicular prosthesis is intraoperatively adjusted in a contactless manner.

Realizing intraoperative adjustability is relatively complex and challenging as viewed upon closer inspection, but is realized according to the invention in a surprisingly simple and effective manner by way of the loops of the adjusting device being made from a material having a memory effect. Each of the loops of at least one of the two partial branches has an activation tab which is mechanically and thermally conductively attached to the outer area of the particular loop that is located radially furthest from the longitudinal axis. The activation tab extends radially away from the loop and has an activation surface oriented perpendicular to the longitudinal axis. The activation surface can induce a thermal activation of the associated loop via the effect of heat. The activation surfaces associated with the loops have a radial separation from the longitudinal axis which successively continuously increases in the axial direction from the first fastening element along the longitudinal axis of the connecting element to the second fastening element. An opening through the perforated top plate of the first fastening element is geometrically designed in such a way that, when the first fastening element is viewed along the longitudinal axis, all the activation surfaces are visible through this opening. Hence, the activation surfaces can be contactlessly heated from the outside by thermal radiation and can thereby activate the associated loops.

In this way, after the ossicular prosthesis according to the invention has been inserted into the middle ear of the patient, its axial length can be contactlessly adjusted, intraoperatively, in an optical manner by radiating energy through the opening in the perforated top plate and onto the activation surface of an activation tab. The emitted energy heats the corresponding activation tab and, finally, the associated loop of the adjusting device is thermally activated via thermal conduction and opens or collapses, depending on the type and configuration of the area made from material having a memory effect.

In embodiments of the invention which are particularly easy to manufacture, the activation tabs are designed in the shape of rectangular platelets having different lengths in the radial direction with respect to the longitudinal axis_{—}

In order to easily reach all the activation surfaces in an optical manner through the opening in the perforated top plate, in the inventive ossicular prosthesis, the activation tabs are arranged in such a way that the successively increasing radial distances of the activation surfaces from the longitudinal axis increment, in the axial direction, in same-sized steps from one loop to the next loop.

In an embodiment, at least one, and preferably multiple connecting segments extend transverse to the longitudinal axis and connect a loop of one partial branch to a loop of a parallel partial branch. As a result, when the adjusting device is pulled apart or compressed, the parallel partial branches are held at an exactly defined distance relative to one another at certain points, and so the adjusting device has an exactly specifiable geometry after it has been plastically deformed.

In an embodiment, in the delivered state of the prosthesis, the loops of the adjusting device are folded tightly together and can be pulled apart by an operating surgeon in the direction of the longitudinal axis of the elongate connecting element in order to attain a desired axial length of the prosthesis. Once the prosthesis has been implanted in the middle ear of the patient, the loops remain, plastically deformed, in this pulled-apart state.

In another embodiment, in the delivered state of the ossicular prosthesis, the loops of the adjusting device are pulled apart and can be compressed by an operating surgeon in the direction of the longitudinal axis of the elongate connecting element in order to attain a desired axial length of the prosthesis. Once the prosthesis has been implanted in the middle ear of the patient, the loops remain, plastically deformed, in this compressed state.

In order to attain an even, defined extension and compression of the adjusting device during plastic deformation, the loops of the adjusting device, which extend transverse to the longitudinal axis of the elongate connecting element, each have the same maximum extension.

Alternatively, axially opposite loop pairs of the adjusting device have maximum extensions which differ transverse to the longitudinal axis of the elongate connecting element, in particular having maximum extensions which continually increase or decrease from one axial end of the adjusting device to the other axial end. It is therefore possible to specify a certain order in which the individual loops become deformed when the adjusting device is extended or compressed. The loops having the greatest maximum extension are typically the softest, in terms of their plastic deformability and therefore deform first when force is applied.

In another embodiment, the adjusting device itself forms the elongate connecting element. Alternatively, the elongate connecting element is designed as a shank, as usual.

Refinements of these embodiments are preferred in which the adjusting device is integrated in the shank, and a connecting piece for the first fastening element and a connecting piece for the second fastening element are located on the two axial ends of the adjusting device, thereby giving the prosthesis a certain level of dimensional stability in the axial direction.

In another embodiment, an engagement device is provided at each of the two axial ends of the adjusting device, preferably axially spaced from the first fastening element and from the second fastening element, at each of which a force-locked or form-locked connection is established using a manipulating instrument, e.g., tweezers or pincers, in order pull apart or compress the adjusting device via the application of force in the direction of the longitudinal axis of the connecting element.

In an embodiment, the second fastening element for the mechanical connection to the head of stapes or the base of stapes is designed as a sleeve, a closed bell, a bell having one or more slots, or a clip. In refinements of this embodiments, the prosthesis is fastened on one side via a top plate to the tympanic membrane and, on the other side, via the second fastening element to the incus or stapes.

Once the prosthesis has been surgically implanted in the middle ear and the tympanic membrane has been closed, the so-called recovery phase begins. Scars form during this period, and they produce unforeseeable forces which can cause the prosthesis to move out of its localized position. When there is a stiff connection between a top plate and the connecting element, increased pressure peaks can result between the edge of the top plate and the tympanic membrane or the graft between the tympanic membrane and the top plate. These pressure peaks can be so high that penetration or extrusion through the tympanic membrane would result. For this reason, it is very helpful for the prosthesis to have a certain amount of post-surgical mobility, so that the top plate can automatically adapt, post surgically, to the position of the tympanic membrane. Since, in addition, the unique anatomical features of the ear, such as the position, shape and size of the stapes, incus, hammer and tympanic membrane vary, it is advantageous when ossicular prostheses are not designed rigid, but rather having a certain amount of flexibility or variability.

In the case of the ossicular prosthesis according to the invention, the connecting element between the two fastening elements is typically designed as an elongate shank, as is well known from the prior art. In order to achieve the increased flexibility and variability of the prosthesis, at least one joint, preferably a ball joint, in particular an axially extending ball joint chain, is provided in the elongate connecting element. In terms of particularly high postsurgical mobility of the prosthesis, refinements are particularly advantageous in which the elongate shank includes a large number of further rotary elements which abut one another, preferably in the form of a ball joint chain.

Embodiments of the invention also are possible in which the prosthesis or parts thereof are made from biocompatible plastics, particularly silicone, polytetrafluoroethylene (PTFE) or polyetheretherketone (PEEK), and/or from fibrous composite materials, in particular carbon fibers. These materials make it possible to prevent postsurgical rejection reactions in most cases.

For that matter, the inventive ossicular prosthesis, or parts thereof, can be made from titanium and/or gold and/or tantalum and/or steel and/or an alloy of the stated metals. It is known that titanium, in particular, in addition to being stiff and having excellent sound-conducting properties, also exhibits excellent biocompatibility with the human middle ear.

In terms of the aforementioned postsurgical position adjustment, embodiments of the invention are advantageous in which parts of the ossicular prosthesis, in particular the loops of the adjusting device, are made from Nitinol. Alternatively, or additionally, parts of the ossicular prosthesis are made from a ceramic material.

In addition to the problem of postsurgical shifting of position, a further problem in the known arts that is overcome by the inventive prostheses results once ossicular prostheses have been implanted, as now described. The middle ear of the human body can be described as a “semi-open region.” Any implantation material that is inserted in the body within the scope of reconstruction of the middle ear and its structures thereby undergoes a particular stress, which predominates in a contaminated and infected environment and which typically attacks the material. Since an objective of implanting an ossicular prosthesis must always be to enable the implant to remain in the patient's middle ear for as long as possible without complications occurring, a sustained attack on the material can result in damage being done to the prosthesis and/or in a local infection. Neither of these consequences is tolerable. In a further embodiment of the invention, in order to permanently prevent damage from occurring to the implantation material or the surrounding tissue, the surface of the ossicular prosthesis is coated entirely or at least in sections with a biologically active coating, in particular a growth-inhibiting and/or growth-promoting and/or antibacterial coating.

In the case of the ossicular prosthesis according to the invention, a fastening element designed as a top plate preferably has a growth-promoting coating, but other parts of the prosthesis should have a growth-inhibiting coating.

According to an embodiment, the mass distribution of the individual parts of the prosthesis is calculated depending on a desired, specified or specifiable frequency response of sound conduction in the middle ear. This allows the sound propagation properties to be mechanically tuned to a certain extent using a custom-made ossicular prosthesis without a great deal of additional technical complexity. In another embodiment, a tuning effect of this type is achieved by fastening at least one additional mass to a part of the ossicular chain or the prosthesis depending on a desired, specifiable frequency response of sound conduction in the middle ear. In a variation of these embodiments, the additional mass is fastened to a part of the ossicular chain or the prosthesis using a clip. The additional mass and/or the clip also can be coated with a biologically active coating.

Finally, another embodiment of the invention is distinguished by the fact that the prosthesis is connected to an active vibrating part of a hearing aid which is active and, in particular, implantable. This also enables further hearing damage caused by the use of modern electronic devices to be largely prevented or at least ameliorated in terms of its effect, and a physical connection of the ossicular prosthesis to the outside world does not cause a problem (due to the coating described above, when the coating is suitably antibacterial), which would result from the increased introduction of bacteria into the region of the middle ear.

The invention also includes a method for thermally activating the loops of an ossicular prosthesis of the above-described type according to the invention, which is distinguished by the fact that the activation surface of an activation tab, whose associated loop is intended to be thermally activated, is radiated, using a laser beam, through the opening in the first fastening element in such a way that the corresponding activation surface heats up. The use of a laser beam for the thermal activation facilitates a particularly precise and fine approach to working during the implantation of the prosthesis.

### BRIEF DESCRIPTION OF THE DRAWINGS

Further features and advantages of the invention will become apparent from the description of embodiments that follows, with reference to the attached figures, wherein:

FIG. 1 depicts a spatial representation of one embodiment of the ossicular prosthesis according to the invention having a perforated tympanic membrane top plate as a first fastening element, an adjusting device having closely folded-together loops, each of which has the same transverse extension, and a piston-shaped, second fastening element;

FIG. 2 depicts a side schematic view of the embodiment from FIG. 1; and

FIG. 3 depicts an axial top view, from above, of the perforated tympanic membrane top plate having the embodiment from FIG. 1 through the opening onto the activation surfaces which have a successively increasing radial separation from the longitudinal axis of the connecting element.

### DETAILED DESCRIPTION OF THE INVENTION

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

An embodiment of the ossicular prosthesis 10 represented schematically in the figures comprises, at one end, a first fastening element 11 that is used for mechanically connecting the prosthesis to the tympanic membrane and is therefore designed as a perforated top plate for placement against the tympanic membrane. Situated at the other end of the ossicular prosthesis 10 is a second fastening element 12 for mechanically connecting the prosthesis to the head of stapes or the base of stapes. Disposed therebetween is an elongate connecting element 13 that interconnects the two fastening elements 11 and 12 in a sound-conducting manner.

The second fastening element 12 is preferably designed in the shape of a piston (as shown). In other embodiments of the inventive ossicular prosthesis that are not shown in the drawing figures, the second fastening element also can be designed differently, e.g., as a slotted bell, a clip, a sleeve, a loop, or a hook. For that matter, the second fastening element also can be designed in the form of a clamp.

The connecting element 13 includes an adjusting device 14 for individually adjusting the axial length of the ossicular prosthesis 10 in the axial direction of the elongate connecting element 13. The first fastening element 11 is mechanically rigidly connected to one end of the connecting element 13 and the second fastening element 12 is mechanically rigidly connected to the other, axially opposite end of the connecting element 13.

The adjusting device 14 includes at least two partial branches which extend symmetrically with respect to the longitudinal axis (a) of the connecting element 13, are extendable and/or compressible in the axial direction, can be permanently plastically deformed in order to fix the axial length of the ossicular prosthesis 10, and are folded, in a serpentine, meandering, or accordion-like manner, into multiple loops 15a′, 15b′, 15c′, 15d, 15a″, 15b″, 15c″, 15d. The multiple loops extend transverse to the longitudinal axis a, before being deformed.

Each partial branch includes at least three loops 15a′, 15b′, 15c′, 15d′ or 15a″, 15b, 15c, 15d″, wherein at least parts of the ossicular prosthesis 10 are made from a material having a memory effect, such as, e.g., Nitinol.

As compared to the prior art, the ossicular prosthesis 10 according to the invention is distinguished by the following features.

Preferably, all the loops 15a′, 15b′, 15c′, 15d′, 15a″, 15b″, 15c″, 15d″ of the adjusting device 14 are made from a material having a memory effect.

Each of the loops 15a′, 15b′, 15c′, 15d′ of at least one of the two partial branches has an activation tab 18a, 18b, 18c, 18d that is mechanically and thermally conductively attached to the outer area of the particular loop that is located radially furthest from the longitudinal axis a, wherein each activation tab 18a, 18b, 18c, 18d extends radially away from the loop 15a′, 15b′, 15c′, 15d′ and has an activation surface 19a, 19b, 19c, 19d oriented perpendicular to the longitudinal axis a. The activation surface can induce a thermal activation of the associated loop 15a′, 15b′, 15c′, 15d′ via the effect of heat. The activation surfaces 19a, 19b, 19c, 19d associated with the loops 15a′, 15b′, 15c′, 15d′ have a radial separation from the longitudinal axis a which successively continuously increases in the axial direction from the first fastening element 11 along the longitudinal axis (a) of the connecting element 13 to the second fastening element 12, in particularly incrementing, in the axial direction, in same-sized steps from one loop 15a′, 15b, 15c′, 15d′ to the next loop.

In the exemplary embodiment depicted, the activation tabs 18a, 18b, 18c, 18d are designed in the shape of rectangular platelets having different lengths in the radial direction with respect to the longitudinal axis a.

Finally, an opening 11′ through the perforated top plate of the first fastening element 11 is geometrically designed in such a way that, when the first fastening element 11 is viewed along the longitudinal axis a, all the activation surfaces 19a, 19b, 19c, 19d are visible through this opening 11′, and so said activation surfaces can be contactlessly heated from the outside by means of thermal radiation, preferably by means of a laser, and can thereby activate the associated loops 15a′, 15b′, 15c′, 15d′.

Moreover, connecting segments 16a, 16b, 16c extending transverse to the longitudinal axis a are provided, each of which connects one loop 15a′, 15b′, 15c′, 15d′ of one partial strand to a loop 15a″, 15b″, 15c″, 15d″ of the parallel partial strand.

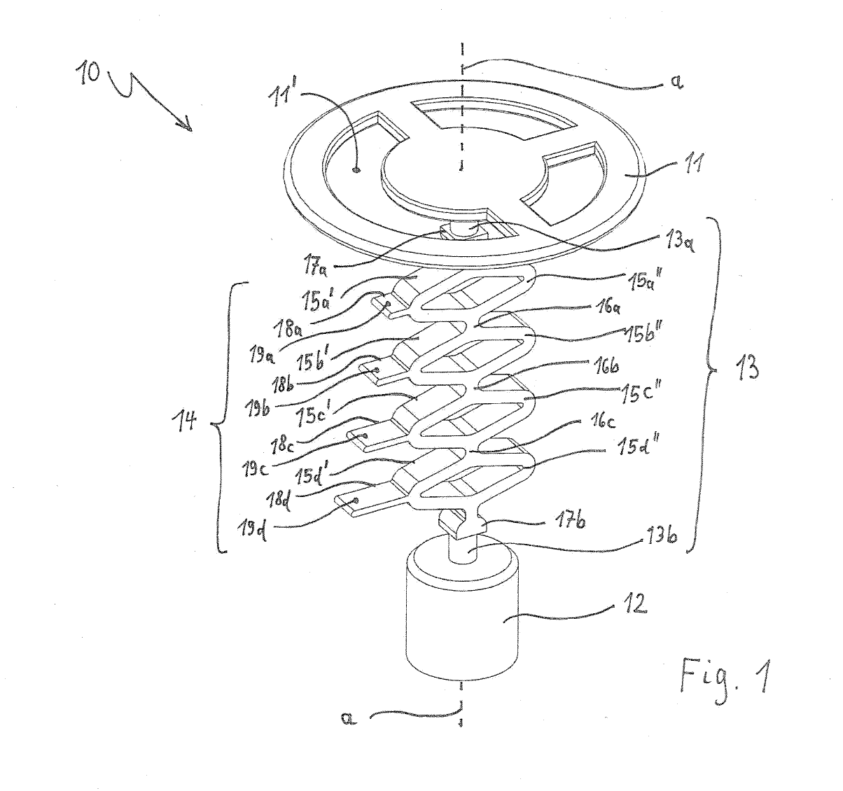

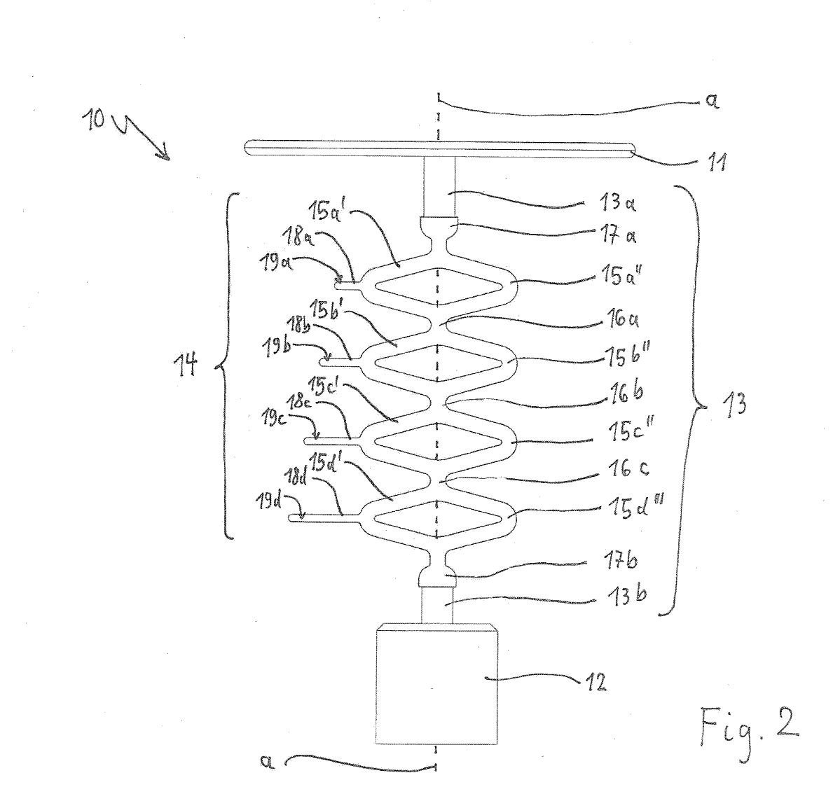

As shown in FIGS. 1 and 2, an engagement device 17a, 17b, is provided at each of the two axial ends of the adjusting device 14 with axial clearance from the first fastening element 11 and from the second fastening element 12, respectively. At each of the engagement devices 17a, 17b, a force-locked or form-locked connection can be established using a manipulating instrument, e.g., tweezers or pincers, in order to pull apart or compress the adjusting device 14 additionally via the application of mechanical force in the direction of the longitudinal axis (a) of the connecting element 13, e.g., in cases in which a laser beam device is unavailable.

As a rule, the elongate connecting element 13 is designed as a two-piece shank, as shown in the figures. In this case, the adjusting device 14 is integrated into the shank. A connecting piece 13a and 13b for the first fastening element 11 and the second fastening element 12, respectively, is located on the two axial ends of the adjusting device 14.

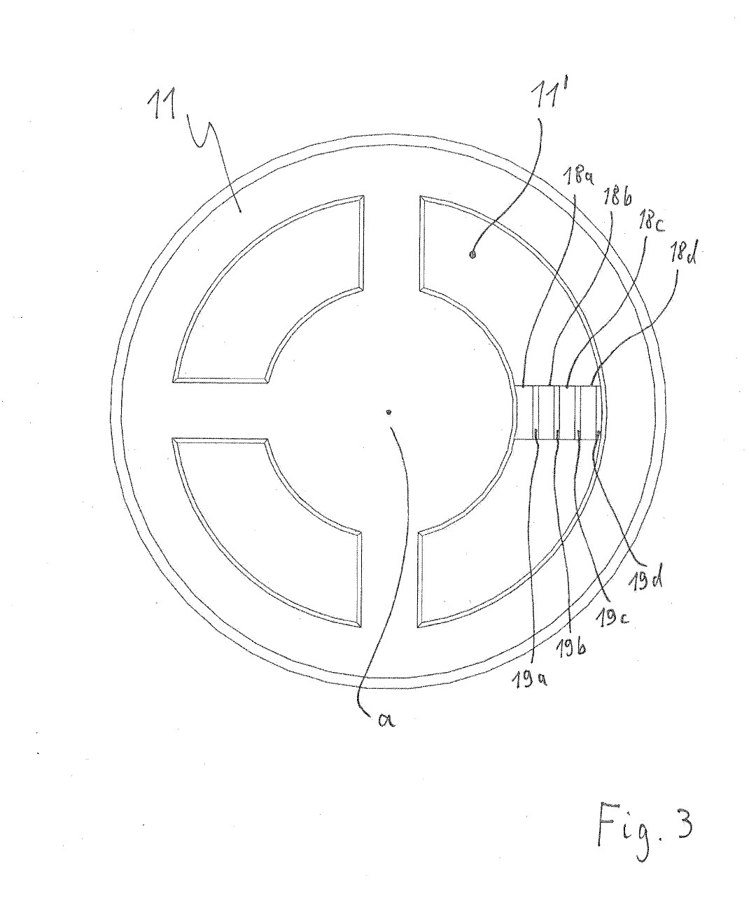

The inventive embodiment depicted in FIGS. 1 to 3 also is distinguished by the fact that the loops 15a′, 15b′, 15c′, 15d′ and 15a, 15b″, 15c″, 15d″ of the adjusting device 14 each have the same maximum extension transverse to the longitudinal axis (a) of the elongate connecting element 13. In embodiments that are not represented in the drawing, axially opposite loop pairs of the adjusting device also can have maximum extensions which differ transverse to the longitudinal axis (a) of the elongate connecting element, in particular having maximum extensions which continuously increase from the top plate-side end of the adjusting device to the other axial end.

While the depicted inventive embodiment is a passive implant, the invention is not limited thereto. The ossicular prosthesis according to the invention also can be designed as part of an active hearing implant system.

With regard to the handling and processing of the ossicular prosthesis according to the invention during implantation, a method for thermally activating the loops 15a′, 15b′, 15c′, 15d′, 15a″, 15b″, 15c″, 15d″ is particularly suitable. According to the inventive method, the activation surface 19a, 19b, 19c, 19d of an activation tab 18a, 18b, 18c, 18d, whose associated loop 15a′, 15b′, 15c′, 15d′, 15a″, 15b″, 15c″, 15d″ is intended to be thermally activated, is radiated, using a laser beam, through the opening 11¹in the first fastening element 11 in such a way that the corresponding activation surface 19a, 19b, 19c, 19d heats up.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. An ossicular prosthesis that replaces or bridges at least one component of the human ossicular chain, comprising:
at one end, a first fastening element designed as a perforated top plate for placement against a tympanic membrane;
at another end, a second fastening element for mechanically connecting the prosthesis to a head of stapes or a base of stapes; and
an elongate connecting element that interconnects the first and the second fastening elements in a sound-conducting manner;
wherein the elongate connecting element includes an adjusting device for adjusting an axial length of the ossicular prosthesis in an axial direction of the elongate connecting element;
wherein the first fastening element is mechanically rigidly connected to one end of the elongate connecting element;
wherein the second fastening element is mechanically rigidly connected to another axially opposite end of the elongate connecting element;
wherein the adjusting device includes at least two partial branches that extend symmetrically with respect to the longitudinal axis of the elongate connecting element, are extendable, compressible or both in the axial direction, are permanently plastically deformable, and are folded into multiple loops transverse to the longitudinal axis in a serpentine, meandering, or accordion-like manner, before being deformed;
wherein parts of the ossicular prosthesis are made from a material having a memory effect;
wherein the loops of the adjusting device are made from a material having a memory effect;
wherein each of the loops of at least one of the two partial branches has an activation tab which is mechanically and thermally conductively attached to an outer area of each particular loop that is located radially furthest from the longitudinal axis;
wherein the activation tab extends radially away from the loop and has an activation surface oriented perpendicular to the longitudinal axis that induces a thermal activation of the associated loop via the effect of heat;
wherein the activation surfaces associated with the loops have a radial separation from the longitudinal axis which successively continuously increases in the axial direction from the first fastening element along the longitudinal axis of the elongate connecting element to the second fastening element;
wherein an opening through the perforated top plate of the first fastening element is geometrically designed in such a way that, when the first fastening element is viewed along the longitudinal axis, all the activation surfaces are visible through the opening; and
wherein the activation surfaces are contactlessly heated from an outside using thermal radiation to activate the associated loops.

2. The ossicular prosthesis according to claim 1, wherein the activation tabs are designed in a shape of rectangular platelets having different lengths in the radial direction, with respect to the longitudinal axis.

3. The ossicular prosthesis according to claim 1, wherein the successively continuously increasing radial distances of the activation surfaces from the longitudinal axis increment, in the axial direction, in same-sized steps from one loop to a next loop.

4. The ossicular prosthesis according to claim 1, wherein connecting segments extending transverse to the longitudinal axis are provided that connect one loop of one partial strand to a loop of a parallel partial strand.

5. The ossicular prosthesis according to claim 1, wherein in a delivered state of the ossicular prosthesis, the loops of the adjusting device are folded tightly together and are pulled apart by an operating surgeon in a direction of the longitudinal axis of the elongate connecting element in order to attain a desired axial length of the prosthesis, and wherein, once the prosthesis is implanted in the middle ear of a patient, the loops remain plastically deformed in the pulled-apart state.

6. The ossicular prosthesis according to claim 1, wherein in a delivered state of the ossicular prosthesis, the loops of the adjusting device are pulled apart, or are compressed by an operating surgeon in a direction of the longitudinal axis of the elongate connecting element to attain a desired axial length of the prosthesis, and wherein once the prosthesis is implanted in the middle ear of the patient, the loops remain, plastically deformed, in the compressed state.

7. The ossicular prosthesis according to claim 1, wherein the loops of the adjusting device each have the same maximum extension transverse to the longitudinal axis of the elongate connecting element.

8. The ossicular prosthesis according to claim 1, wherein axially opposite loop pairs of the adjusting device have different maximum extensions transverse to the longitudinal axis of the elongate connecting element.

9. The ossicular prosthesis according to claim 1, wherein the adjusting device forms the elongate connecting element.

10. The ossicular prosthesis according to claim 1, wherein the elongate connecting device is designed as a shank, wherein the adjusting device is integrated into the shank and wherein a connecting piece for the first fastening element and a connecting piece for the second fastening element are located on the two axial ends of the adjusting device.

11. The ossicular prosthesis according to claim 1, wherein the connecting element comprises at least one joint.

12. The ossicular prosthesis according to claim 1, wherein the second fastening element for the mechanical connection to the head of stapes or the base of stapes is designed as any of the following: a sleeve, a closed bell, a bell having one or more slots and a clip.

13. The ossicular prosthesis according to claim 1, wherein one or more parts of the ossicular prosthesis are made from Nitinol.

14. An active hearing system comprising the ossicular prosthesis according to claim 1.

15. A method for thermally activating the loops of an ossicular prosthesis according to claim 1, the method including a step of radiating an activation surface of an activation tab, whose associated loop is intended to be thermally activated using a laser beam.

16. The method according to claim 16, wherein the activation surface is radiated through the opening in the first fastening element in such a way that the corresponding activation surface heats up.
